(19) United States
(12) Patent Application Publication
(10) Pub. No.: US 2006/0287392 A1
(43) Pub. Date: Dec. 21, 2006

(54) GABAPENTIN COMPOSITIONS

(75) Inventors: Matthew Peterson, Hopkinton, MA (US); Mark Oliveira, Framingham, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: TRANSFORM PHARMACEUTICALS, INC., Lexington, MA (US)

(21) Appl. No.: 11/461,830

(22) Filed: Aug. 2, 2006

Related U.S. Application Data
(62) Division of application No. 10/820,382, filed on Apr. 8, 2004.
(60) Provisional application No. 60/462,179, filed on Apr. 11, 2003.

(51) Int. Cl. A61K 31/195; C07C 229/28; C07C 275/02

(52) U.S. Cl. 514/561; 514/588; 562/504

(57) ABSTRACT

The present invention comprises an organic acid salt of gabapentin, wherein the organic acid is tartaric acid, ethanedisulfonic acid, or maleic acid. Methods for modulating the solubility and dose response of gabapentin are discussed. Methods of making organic acid salts of gabapentin are also discussed.

### RELATED APPLICATIONS

This application claims priority to U.S. Provisional Application No. 60/462,179 filed on Apr. 11, 2003 which is hereby incorporated by reference.

### FIELD OF THE INVENTION

The present invention relates to drug-containing compositions, pharmaceutical compositions comprising such drugs, and methods for preparing same.

### BACKGROUND OF THE INVENTION

Drugs in pharmaceutical compositions can be prepared in a variety of different forms. Such drugs can be prepared so as to have a variety of different chemical forms including chemical derivatives or salts. Such drugs can also be prepared to have different physical forms. For example, the drugs may be amorphous or may have different crystalline polymorphs, perhaps existing in different solvation or hydration states. By varying the form of a drug, it is possible to vary the physical properties thereof. For example, crystalline polymorphs typically have different solubilities from one another, such that a more thermodynamically stable polymorph is less soluble than a less thermodynamically stable polymorph. Pharmaceutical polymorphs can also differ in properties such as shelf-life, bioavailability, morphology, vapor pressure, density, color, and compressibility.

Gabapentin is 1-(aminomethyl)-1-cyclohexane acetic acid which is represented by the structure:

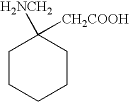

Gabapentin is indicated as an anticonvulsant and is used in the treatment of diseases of the brain, including epilepsy. Various synthetic routes leading to different polymorphs of gabapentin are reported in U.S. Pat. No. 4,024,175, U.S. Pat. No. 4,894,476 and U.S. Pat. No. 6,255,526. U.S. Pat. No. 4,024,175 relates generally to cyclic amino acids and discusses the possibility of making pharmacologically compatible salts with amino acids, on account of their amphotericity. Among such salts are mentioned salts of inorganic and organic acids, as well as salts with alkali metals, alkaline earth metals, and quarternary ammonium ions. Included in the disclosure is a hydrochloride salt of gabapentin, as well as the sodium, calcium and ammonium salts thereof

U.S. Pat. No. 4,894,476 suggests that the compounds of U.S. Pat. No. 4,024,175 are relatively expensive and provides a monohydrate form of gabapentin which is apparently less expensive to produce and has marginally detectable solvent residues. A process is provided for manufacturing the monohydrate form of gabapentin that starts from a salt form which is preferably the hydrochloride. This salt form is treated in a number of steps so as to be converted to crystalline gabapentin monohydrate.

U.S. Pat. No. 6,255,526 describes a method of converting gabapentin hydrochloride to gabapentin form II. This patent indicates that prior art methods such as described in U.S. Pat. No. 4,024,175 and U.S. Pat. No. 4,894,476 are industrially impractical.

It would be advantageous to have new forms of gabapentin that have improved properties, in particular, as oral formulations. Specifically, it is desirable to identify improved forms of the drug that exhibit significantly increased aqueous solubilities. It is also desirable to increase the dissolution rate of drug-containing pharmaceutical compositions in water, increase the bioavailability of orally-administered compositions, and provide a more rapid onset to therapeutic effect. It is also desirable to have a form of the drug which, when administered to a subject, reaches a peak plasma level faster and/or has a longer lasting plasma concentration and higher overall exposure at high doses when compared to equivalent amounts of the drug in its presently-known form.

### SUMMARY OF THE INVENTION

It has now been found that new salt forms of gabapentin can be obtained which have improved properties as compared to the converted forms of the drug.

Accordingly, in a first aspect, the present invention provides an organic acid salt of gabapentin, wherein the organic acid is tartaric acid, ethanedisulfonic acid, or maleic acid.

It has surprisingly been found that when gabapentin and a selected organic acid are allowed to form salts, the resulting salts give rise to improved properties of the drug, particularly with respect to solubility properties, such as aqueous solubility, and dose response properties. This is particularly advantageous where the original drug is sparingly soluble in water. Additionally, the properties which may be conferred upon the drug are useful because the bioavailability of the drug can be improved and the plasma concentration and/or serum concentration of the drug can be improved. This is particularly advantageous for orally-administrable formulations. Moreover, the dose response of the drug can be improved, for example by increasing the maximum attainable response and/or increasing the potency of the drug by increasing the biological activity per dosing equivalent.

Where the organic acid is tartaric acid, the mole ratio of gabapentin to tartaric acid is typically approximately 1:1. Alternatively, where the organic acid is ethanedisulfonic acid, the mole ratio of gabapentin to ethanedisulfonic acid is typically approximately 2:1. These mole ratios are found when the gabapentin salt is prepared according to methods described herein. Other mole ratios may be possible. The physical form of the organic acid salt is preferably compatible with its ability to be readily formed as a pharmaceutical composition. It is preferred that the organic acid salt is in a crystalline form and such crystalline forms are readily preparable according to the methods described herein.

The invention further provides a pharmaceutical composition comprising a tartaric acid, ethanedisulfonic acid, or maleic acid salt of gabapentin. Typically, the pharmaceutical composition further comprises one or more pharmaceutically-acceptable carriers, diluents or excipients. Pharmaceutical compositions according to the invention are described in further detail below.

In a further aspect, the present invention provides a process for the preparation of a tartaric acid, ethanedisulfonic acid, or maleic acid salt of gabapentin, which comprises:
(1) mixing gabapentin with an organic acid to form a mixture;
(2) subjecting the mixture to conditions which salify the gabapentin whereby crystals of a gabapentin salt are formed; and
(3) optionally isolating the salt, wherein the organic acid is tartaric acid, ethanedisulfonic acid, or maleic acid.

In another aspect, the gabapentin may be mixed with the organic acid in solution. Any suitable solvent may be used for this step, including organic solvents or mixed solvents. Solvents comprising alcohols can be used; Methanol is a preferred alcohol. A water/methanol mixed solvent can also be used.

Any conditions which salify the gabapentin from solution may be used whereby crystals of the gabapentin salt are formed. Conveniently, this step includes evaporation of the solvent so as to concentrate the solute whereby gabapentin salt crystals may be precipitated. In a preferred embodiment, the solution is first heated to ensure mixing and salt formation, followed by cooling so as to enable salt crystals to precipitate.

In an alternative embodiment, the gabapentin is mixed with the organic acid in a solid phase. Any suitable means for mixing may be used in this step, including commercially-available solid mixers. The solid mixture thus formed is preferably heated so as to cause salification of the gabapentin with the organic acid. In this step it is possible that salt crystals may form spontaneously upon heating. It is preferred in this embodiment to ensure that the solid mixture is comminuted, typically by grinding the mixture prior to heating so as to facilitate salification.

The salt, typically in the form of crystals, may be isolated by any conventional technique.

In a further aspect, the present invention provides a process for modulating the solubility of gabapentin for use in a pharmaceutical composition, which process comprises:
(1) mixing gabapentin with an organic acid to form a mixture; and
(2) salifying the gabapentin with the organic acid so that the solubility of the gabapentin is modulated, wherein the organic acid is tartaric acid, ethanedisulfonic acid, or maleic acid.

In a further aspect, the present invention provides a process for modulating the dose response of gabapentin for use in a pharmaceutical composition, which process comprises:
(1) mixing gabapentin with an organic acid to form a mixture, and
(2) salifying the gabapentin with the organic acid so that the dose response of the gabapentin is modulated, wherein the organic acid is tartaric acid, ethanedisulfonic acid, or maleic acid.

The processes according to the present invention may each comprise a further step or steps in which the gabapentin salt produced thereby is incorporated into a pharmaceutical composition.

In a still further aspect of the invention, a method is provided for treating a subject, preferably a human subject, with a brain disorder. The method comprises administering to the subject a therapeutically-effective amount of a tartaric acid, ethanedisulfonic acid, or maleic acid salt of gabapentin. Such brain disorders include epilepsy, mood disorders, and other disorders susceptible to treatment with an anticonvulsant.

The invention will now be described in further detail, by way of example only, with reference to the accompanying drawings.

### BRIEF DESCRIPTION OF THE DRAWINGS

FIG. 1 shows PXRD diffractograms for gabapentin form 1, a mixture of gabapentin monohydrate and form 1, and gabapentin DL-tartaric acid salt;

FIG. 2 shows a PXRD diffractogram of a mixture of gabapentin and DL-tartaric acid;

FIG. 3 shows a DSC thermogram of a mixture of gabapentin and DL-tartaric acid;

FIG. 4 shows a PXRD diffractogram for the gabapentin DL-tartaric acid salt;

FIG. 5 shows a DSC thermogram for the gabapentin DL-tartaric acid salt;

FIG. 6 shows a TGA thermogram for the gabapentin DL-tartaric acid salt;

FIG. 7 shows PXRD diffractograms for ethanedisulfonic acid, a mixture of gabapentin monohydrate and gabapentin form 1, gabapentin form 1 and gabapentin ethanedisulfonic acid salt;

FIG. 8 shows a PXRD diffractogram for a solid resulting from grinding gabapentin and ethanedisulfonic acid solids;

FIG. 9 shows a DSC thermogram for the solid resulting from grinding gabapentin and ethanedisulfonic acid solids;

FIG. 10 shows a PXRD diffractogram for the gabapentin ethanedisulfonic acid salt;

FIG. 11 shows a DSC thermogram for gabapentin ethanedisulfonic acid salt;

FIG. 12 shows a TGA thermogram for the gabapentin ethanedisulfonic acid salt;

FIG. 13 shows a PXRD diffractogram for the gabapentin ethanedisulfonic acid salt;

FIG. 14 shows a PXRD diffractogram for gabapentin maleic acid salt;

FIG. 15 shows a DSC thermogram for gabapentin maleic acid salt;

FIG. 16 shows a DSC thermogram for gabapentin:urea co-crystal;

FIG. 17 shows a TGA thermogram for gabapentin:urea co-crystal;

FIG. 18 shows a PXRD diffractogram for gabapentin:urea co-crystal.

### DETAILED DESCRIPTION OF THE INVENTION

Excipients employed in pharmaceutical compositions of the present invention can be solids, semi-solids, liquids or combinations thereof. Preferably, excipients are solids. Compositions of the invention containing excipients can be prepared by any known technique of pharmacy that comprises admixing an excipient with a drug or therapeutic agent. A pharmaceutical composition of the invention contains a desired amount of drug per dose unit and, if intended for oral administration, can be in the form, for example, of a tablet, a caplet, a pill, a hard or soft capsule, a lozenge, a cachet, a dispensable powder, granules, a suspension, an elixir, a dispersion, a liquid, or any other form reasonably adapted for such administration. If intended for parenteral administration, it can be in the form, for example, of a suspension or transdermal patch. If intended for rectal administration, it can be in the form, for example, of a suppository. Presently preferred are oral dosage forms that are discrete dose units each containing a predetermined amount of the drug, such as tablets or capsules.

Non-limiting examples follow of excipients that can be used to prepare pharmaceutical compositions of the invention.

Pharmaceutical compositions of the invention optionally comprise one or more pharmaceutically acceptable carriers or diluents as excipients. Suitable carriers or diluents illustratively include, but are not limited to, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of alpha- and amorphous cellulose (e.g., RexcelJ), powdered cellulose, hydroxypropylcellulose (HPC) and hydroxypropylmethylcellulose (HPMC); calcium carbonate; glycine; bentonite; block co-polymers; polyvinylpyrrolidone; and the like. Such carriers or diluents, if present, constitute in total about 5% to about 99%, preferably about 10% to about 85%, and more preferably about 20% to about 80%, of the total weight of the composition. The carrier, carriers, diluent, or diluents selected preferably exhibit suitable flow properties and, where tablets are desired, compressibility.

Lactose, mannitol, dibasic sodium phosphate, and microcrystalline cellulose (particularly Avicel PH microcrystalline cellulose such as Avicel PH 101), either individually or in combination, are preferred diluents. These diluents are chemically compatible with drugs. The use of extragranular microcrystalline cellulose (that is, microcrystalline cellulose added to a granulated composition) can be used to improve hardness (for tablets) and/or disintegration time. Lactose, especially lactose monohydrate, is particularly preferred. Lactose typically provides compositions having suitable release rates of drugs, stability, pre-compression flowability, and/or drying properties at a relatively low diluent cost. It provides a high density substrate that aids densification during granulation (where wet granulation is employed) and therefore improves blend flow properties and tablet properties.

Pharmaceutical compositions of the invention optionally comprise one or more pharmaceutically acceptable disintegrants as excipients, particularly for tablet formulations. Suitable disintegrants include, but are not limited to, either individually or in combination, starches, including sodium starch glycolate (e.g., Explotab™ of PenWest) and pregelatinized corn starches (e.g., National™ 1551 of National Starch and Chemical Company, National™ 1550, and Coloccom™ 1500), clays (e.g., Veegum™ HV of R. T. Vanderbilt), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, locust bean, karaya, pectin and tragacanth gums.

Disintegrants may be added at any suitable step during the preparation of the composition, particularly prior to granulation or during a lubrication step prior to compression. Such disintegrants, if present, constitute in total about 0.2% to about 30%, preferably about 0.2% to about 10%, and more preferably about 0.2% to about 5%, of the total weight of the composition.

Croscarmellose sodium is a preferred disintegrant for tablet or capsule disintegration, and, if present, preferably constitutes about 0.2% to about 10%, more preferably about 0.2% to about 7%, and still more preferably about 0.2% to about 5%, of the total weight of the composition. Croscarmellose sodium confers superior intragranular disintegration capabilities to granulated pharmaceutical compositions of the present invention.

Pharmaceutical compositions of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Such binding agents may also prevent or inhibit crystallization or recrystallization of a drug of the present invention once the salt has been dissolved in a solution. Suitable binding agents and adhesives include, but are not limited to, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel™ of Aqualon); and ethylcellulose (e.g., Ethocel™ of the Dow Chemical Company). Such binding agents and/or adhesives, if present, constitute in total about 0.5% to about 25%, preferably about 0.75% to about 15%, and more preferably about 1% to about 10%, of the total weight of the pharmaceutical composition.

Many of the binding agents are polymers comprising amide, ester, ether, alcohol or ketone groups and, as such, are preferably included in pharmaceutical compositions of the present invention. Polyvinylpyrrolidones such as povidone K-30 are especially preferred. Polymeric binding agents can have varying molecular weight, degrees of crosslinking, and grades of polymer. Polymeric binding agents can also be copolymers, such as block co-polymers that contain mixtures of ethylene oxide and propylene oxide units. Variation in these units' ratios in a given polymer affects properties and performance. Examples of block co-polymers with varying compositions of block units are Poloxamer 188 and Poloxamer 237 (BASF Corporation).

Pharmaceutical compositions of the invention optionally comprise one or more pharmaceutically acceptable wetting agents as excipients. Such wetting agents are preferably selected to maintain the drug in close association with water, a condition that is believed to improve bioavailability of the composition.

Non-limiting examples of surfactants that can be used as wetting agents in pharmaceutical compositions of the invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefosse), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefosse), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, preferably about 0.4% to about 10%, and more preferably about 0.5% to about 5%, of the total weight of the pharmaceutical composition.

Wetting agents that are anionic surfactants are preferred. Sodium lauryl sulfate is a particularly preferred wetting agent. Sodium lauryl sulfate, if present, constitutes about 0.25% to about 7%, more preferably about 0.4% to about 4%, and still more preferably about 0.5% to about 2%, of the total weight of the pharmaceutical composition.

Pharmaceutical compositions of the invention optionally comprise one or more pharmaceutically acceptable lubricants (including anti-adherents and/or glidants) as excipients. Suitable lubricants include, but are not limited to, either individually or in combination, glyceryl behapate (e.g., Compritol™ 888 of Gattefosse); stearic acid and salts thereof, including magnesium, calcium and sodium stearates; hydrogenated vegetable oils (e.g., Sterotex™ of Abitec); colloidal silica; talc; waxes; boric acid; sodium benzoate; sodium acetate; sodium fumarate; sodium chloride; DL-leucine; PEG (e.g., Carbowax™ 4000 and Carbowax™ 6000 of the Dow Chemical Company); sodium oleate; sodium lauryl sulfate; and magnesium lauryl sulfate. Such lubricants, if present, constitute in total about 0.1% to about 10%, preferably about 0.2% to about 8%, and more preferably about 0.25% to about 5%, of the total weight of the pharmaceutical composition.

Magnesium stearate is a preferred lubricant used, for example, to reduce friction between the equipment and granulated mixture during compression of tablet formulations.

Suitable anti-adherents include, but are not limited to, talc, cornstarch, DL-leucine, sodium lauryl sulfate and metallic stearates. Talc is a preferred anti-adherent or glidant used, for example, to reduce formulation sticking to equipment surfaces and also to reduce static in the blend. Talc, if present, constitutes about 0. 1% to about 10%, more preferably about 0.25% to about 5%, and still more preferably about 0.5% to about 2%, of the total weight of the pharmaceutical composition.

Glidants can be used to promote powder flow of a solid formulation. Suitable glidants include, but are not limited to, colloidal silicon dioxide, starch, talc, tribasic calcium phosphate, powdered cellulose and magnesium trisilicate. Colloidal silicon dioxide is particularly preferred.

Other excipients such as colorants, flavors and sweeteners are known in the pharmaceutical art and can be used in pharmaceutical compositions of the present invention. Tablets can be coated, for example with an enteric coating, or uncoated. Compositions of the invention can further comprise, for example, buffering agents.

Optionally, one or more effervescent agents can be used as disintegrants and/or to enhance organoleptic properties of pharmaceutical compositions of the invention. When present in pharmaceutical compositions of the invention to promote dosage form disintegration, one or more effervescent agents are preferably present in a total amount of about 30% to about 75%, and preferably about 45% to about 70%, for example about 60%, by weight of the pharmaceutical composition.

According to a particularly preferred embodiment of the invention, an effervescent agent, present in a solid dosage form in an amount less than that effective to promote disintegration of the dosage form, provides improved dispersion of the drug in an aqueous medium. Without being bound by theory, it is believed that the effervescent agent is effective to accelerate dispersion of the drug, from the dosage form in the gastrointestinal tract, thereby further enhancing absorption and rapid onset of therapeutic effect. When present in a pharmaceutical composition of the invention to promote intragastrointestinal dispersion but not to enhance disintegration, an effervescent agent is preferably present in an amount of about 1% to about 20%, more preferably about 2.5% to about 15%, and still more preferably about 5% to about 10%, by weight of the pharmaceutical composition.

An “effervescent agent” herein is an agent comprising one or more compounds which, acting together or individually, evolve a gas on contact with water. The gas evolved is generally oxygen or, most commonly, carbon dioxide. Preferred effervescent agents comprise an acid and a base that react in the presence of water to generate carbon dioxide gas. Preferably, the base comprises an alkali metal or alkaline earth metal carbonate or bicarbonate and the acid comprises an aliphatic carboxylic acid.

Non-limiting examples of suitable bases as components of effervescent agents useful in the invention include carbonate salts (e.g., calcium carbonate), bicarbonate salts (e.g., sodium bicarbonate), sesquicarbonate salts, and mixtures thereof. Calcium carbonate is a preferred base.

Non-limiting examples of suitable acids as components of effervescent agents and/or solid organic acids useful in the invention include citric acid, tartaric acid (as D-, L-, or DL-tartaric acid), malic acid, maleic acid, fumaric acid, adipic acid, succinic acid, acid anhydrides of such acids, acid salts of such acids, and mixtures thereof. Citric acid is a preferred acid.

In a preferred embodiment of the invention, where the effervescent agent comprises an acid and a base, the weight ratio of the acid to the base is about 1:100 to about 100:1, more preferably about 1:50 to about 50:1, and still more preferably about 1:10 to about 10:1. In a further preferred embodiment of the invention, where the effervescent agent comprises an acid and a base, the ratio of the acid to the base is approximately stoichiometric.

Excipients which solubilize metal salts of drugs typically have both hydrophilic and hydrophobic regions, or are preferably amphiphilic or have amphiphilic regions. One type of amphiphilic or partially-amphiphilic excipient comprises an amphiphilic polymer or is an amphiphilic polymer. A specific amphiphilic polymer is a polyalkylene glycol, which is commonly comprised of ethylene glycol and/or propylene glycol subunits. Such polyalkylene glycols can be esterified at their termini by a carboxylic acid, ester, acid anhyride or other suitable moiety. Examples of such excipients include poloxamers (symmetric block copolymers of ethylene glycol and propylene glycol; e.g., poloxamer 237), polyalkyene glycolated esters of tocopherol (including esters formed from a di- or multi-functional carboxylic acid; e.g., d-alpha-tocopherol polyethylene glycol-1000 succinate), and macrogolglycerides (formed by alcoholysis of an oil and esterification of a polyalkylene glycol to produce a mixture of mono-, di- and tri-glycerides and mono- and di-esters; e.g., stearoyl macrogol-32 glycerides). Such pharmaceutical compositions are advantageously administered orally.

Pharmaceutical compositions of the present invention can comprise about 10% to about 50%, about 25% to about 50%, about 30% to about 45%, or about 30% to about 35% by weight of drug; about 10% to about 50%, about 25% to about 50%, about 30% to about 45%, or about 30% to about 35% by weight of a an excipient which inhibits crystallization; and about 5% to about 50%, about 10% to about 40%, about 15% to about 35%, or about 30% to about 35% by weight of a binding agent. In one example, the weight ratio of the drug to the excipient which inhibits crystallization to binding agent is about 1 to 1 to 1.

Solid dosage forms of the invention can be prepared by any suitable process, not limited to processes described herein.

An illustrative process comprises (a) a step of blending a salt of the invention with one or more excipients to form a blend, and (b) a step of tableting or encapsulating the blend to form tablets or capsules, respectively.

In a preferred process, solid dosage forms are prepared by a process comprising (a) a step of blending a drug salt of the invention with one or more excipients to form a blend, (b) a step of granulating the blend to form a granulate, and (c) a step of tableting or encapsulating the blend to form tablets or capsules respectively. Step (b) can be accomplished by any dry or wet granulation technique known in the art, but is preferably a dry granulation step. A salt of the present invention is advantageously granulated to form particles of about 1 micrometer to about 100 micrometer, about 5 micrometer to about 50 micrometer, or about 10 micrometer to about 25 micrometer. One or more diluents, one or more disintegrants and one or more binding agents are preferably added, for example in the blending step, a wetting agent can optionally be added, for example in the granulating step, and one or more disintegrants are preferably added after granulating but before tableting or encapsulating. A lubricant is preferably added before tableting. Blending and granulating can be performed independently under low or high shear. A process is preferably selected that forms a granulate that is uniform in drug content, that readily disintegrates, that flows with sufficient ease so that weight variation can be reliably controlled during capsule filling or tableting, and that is dense enough in bulk so that a batch can be processed in the selected equipment and individual doses fit into the specified capsules or tablet dies.

In an alternative embodiment, solid dosage forms are prepared by a process that includes a spray drying step, wherein the drug is suspended with one or more excipients in one or more sprayable liquids, preferably a non-protic (e.g., non-aqueous or non-alcoholic) sprayable liquid, and then is rapidly spray dried over a current of warm air.

A granulate or spray dried powder resulting from any of the above illustrative processes can be compressed or molded to prepare tablets or encapsulated to prepare capsules. Conventional tableting and encapsulation techniques known in the art can be employed. Where coated tablets are desired, conventional coating techniques are suitable.

Excipients for tablet compositions of the invention are preferably selected to provide a disintegration time of less than about 30 minutes, preferably about 25 minutes or less, more preferably about 20 minutes or less, and still more preferably about 15 minutes or less, in a standard disintegration assay.

Gabapentin dosage forms of the invention preferably comprise the gabapentin salt in a daily dosage amount of about 900 to 1800 mg and given in divided doses (three times a day) using 300, 400, 600 or 800 mg tablets/capsules. The starting dose is usually about 300 mg three times a day, but will vary depending on the indication and specific patient.

Pharmaceutically acceptable salts of gabapentin can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherngju, Controlled Release Dosage Form Design, 2 Technomic Publishing, Lancaster, Pa.: 2000).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the gabapentin salts and compositions of the invention. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of gabapentin and thus effect controlled delivery of the drug. Examples of specific anion exchangers include, but are not limited to, Duolite® A568 and Duolite® AP143 (Rohm & Haas, Spring House, Pa. USA).

One embodiment of the invention encompasses a unit dosage form which comprises a pharmaceutically acceptable salt of gabapentin (e.g., a tartaric acid salt), or a polymorph, solvate, hydrate, dehydrate, co-crystal, anhydrous, or amorphous form thereof, and one or more pharmaceutically acceptable excipients or diluents, wherein the pharmaceutical composition or dosage form is formulated for controlled-release. Specific dosage forms utilize an osmotic drug delivery system.

A particular and well-known osmotic drug delivery system is referred to as OROS® (Alza Corporation, Mountain View, Calif. USA). This technology can readily be adapted for the delivery of compounds and compositions of the invention. Various aspects of the technology are disclosed in U.S. Pat. Nos. 6,375,978 B1; 6,368,626 B1; 6,342,249 B1; 6,333,050 B2; 6,287,295 B1; 6,283,953 B1; 6,270,787 B1; 6,245,357 B1; and 6,132,420; each of which is incorporated herein by reference. Specific adaptations of OROS® that can be used to administer compounds and compositions of the invention include, but are not limited to, the OROS® Push-Pull™, Delayed Push-Pull™, Multi-Layer Push-Pull™, and Push-Stick™ Systems, all of which are well known. See, e.g., http://www.alza.com. Additional OROS® systems that can be used for the controlled oral delivery of compounds and compositions of the invention include OROS®-CT and L-OROS®. Id.; see also, Delivery Times, vol. II, issue II (Alza Corporation).

Conventional OROS® oral dosage forms are made by compressing a drug powder (e.g., gabapentin salt) into a hard tablet, coating the tablet with cellulose derivatives to form a semi-permeable membrane, and then drilling an orifice in the coating (e.g., with a laser). (Kim, Cherng-ju, Controlled Release Dosage Form Design, 231-238 Technomic Publishing, Lancaster, Pa.: 2000). The advantage of such dosage forms is that the delivery rate of the drug is not influenced by physiological or experimental conditions. Even a drug with a pH-dependent solubility can be delivered at a constant rate regardless of the pH of the delivery medium. But because these advantages are provided by a build-up of osmotic pressure within the dosage form after administration, conventional OROS® drug delivery systems cannot be used to effectively deliver drugs with low water solubility.

A specific dosage form of the invention comprises: a wall defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent to the exit orifice and in direct or indirect contacting relationship with the expandable layer; and a flow-promoting layer interposed between the inner surface of the wall and at least the external surface of the drug layer located within the cavity, wherein the drug layer comprises a salt of gabapentin, or a polymorph, solvate, hydrate, dehydrate, co-crystal, anhydrous, or amorphous form thereof See U.S. Pat. No. 6,368,626, the entirety of which is incorporated herein by reference.

Another specific dosage form of the invention comprises: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent formulation comprises a salt of gabapentin, or a polymorph, solvate, hydrate, dehydrate, co-crystal, anhydrous, or amorphous form thereof. See U.S. Pat. No. 6,342,249, the entirety of which is incorporated herein by reference.

### EXAMPLES

Analytical Methods

DSC analysis of the samples was performed using a Q1000 Differential Scanning Calorimeter (TA Instruments, New Castle, Del., U.S.A.), which uses Advantage for QW-Series, version 1.0.0.78, Thermal Advantage Release 2.0 (2001 TA Instruments-Water LLC). In addition, the analysis software used was Universal Analysis 2000 for Windows 95/95/2000/NT, version 3.1E;Build 3.1.0.40 (2001 TA Instruments-Water LLC).

For the DSC analysis, the purge gas used was dry nitrogen, the reference material was an empty aluminum pan that was crimped, and the sample purge was 50 mL/minute.

DSC analysis of the sample was performed by placing 2.594 mg of sample in an aluminum pan with a crimped pan closure. The starting temperature was typically 20° C. with a heating rate of 10° C./minute, and the ending temperature was 200° C.

TGA analysis of samples was performed using a Q500 Thermogravimetric Analyzer (TA Instruments, New Castle, Del., U.S.A.), which uses Advantage for QW-Series, version 1.0.0.78, Thermal Advantage Release 2.0 (2001 TA Instruments-Water LLC). In addition, the analysis software used was Universal Analysis 2000 for Windows 95/95/2000/NT, version 3. IE;Build 3.1.0.40 (2001 TA Instruments-Water LLC).

For all of the TGA experiments, the purge gas used was dry nitrogen, the balance purge was 40 mL/minute N₂, and the sample purge was 60 mL/minute N₂.

TGA of the sample was performed by placing 2.460 mg of sample in a platinum pan. The starting temperature was typically 20° C. with a heating rate of 10° C./minute, and the ending temperature was 300° C.

A powder X-ray diffraction pattern for the samples was obtained using a D/Max Rapid, Contact (Rigaku/MSC, The Woodlands, Tex., U.S.A.), which uses as its control software RINT Rapid Control Software, Rigaku Rapid/XRD, version 1.0.0 (1999 Rigaku Co.). In addition, the analysis software used were RINT Rapid display software, version 1.18 (Rigaku/MSC), and JADE XRD Pattern Processing, versions 5.0 and 6.0 ((1995-2002, Materials Data, Inc.).

For the PXRD analysis, the acquisition parameters were as follows: source was Cu with a K line at 1.5406 Å; x-y stage was manual; collimator size was 0.3 mm; capillary tube (Charles Supper Company, Natick, Mass., U.S.A.) was 0.3 mm ID; reflection mode was used; the power to the X-ray tube was 46 kV; the current to the X-ray tube was 40 mA; the omega-axis was oscillating in a range of 0-5 degrees at a speed of 1 degree/minute; the phi-axis was spinning at an angle of 360 degrees at a speed of 2 degrees/second; 0.3 mm collimator; the collection time was 60 minutes; the temperature was room temperature; and the heater was not used. The sample was presented to the X-ray source in a boron rich glass capillary.

In addition, the analysis parameters were as follows: the integration 2-theta range was 2-60 degrees; the integration chi range was 0-360 degrees; the number of chi segments was 1; the step size used was 0.02; the integration utility was cylint; normalization was used; dark counts were 8; omega offset was 180; and chi and phi offsets were 0.

The relative intensity of peaks in a diffractogram is not necessarily a limitation of the PXRD pattern because peak intensity can vary from sample to sample, e.g., due to crystalline impurities. Further, the angles of each peak can vary by about ±0.1 degrees, preferably ±0.05. The entire pattern or most of the pattern peaks may also shift by about ±0.1 degree due to differences in calibration, settings, and other variations from instrument to instrument and from operator to operator. The above limitations result in a PXRD error of about ±0.1 degrees 2-theta for each diffraction peak.

Single crystal x-ray data were collected on a Bruker SMART-APEX CCD diffractometer (M. J. Zaworotko, Department of Chemistry, University of South Florida). Lattice parameters were determined from least squares analysis. Reflection data was integrated using the program SAINT. The structure was solved by direct methods and refined by full matrix least squares using the program SHELXTL (Sheldrick, G. M. SHELXTL, Release 5.03; Siemans Analytical X-ray Instruments Inc.: Madison, Wis.).

For PXRD data below and in the Figures, each composition of the present invention may be characterized by any one, any two, any three, any four, any five, any six, any seven, any eight or more of the 2 theta angle peaks. For DSC data below and in the Figures, each composition of the present invention may be characterized by any one, any two, any three, or more endothermic and/or exothermic transitions. Single crystal x-ray analysis may also be used to characterize a composition of the present invention.

### Example 1

### Preparation of Gabapentin DL-Tartaric Acid Salt from Solution

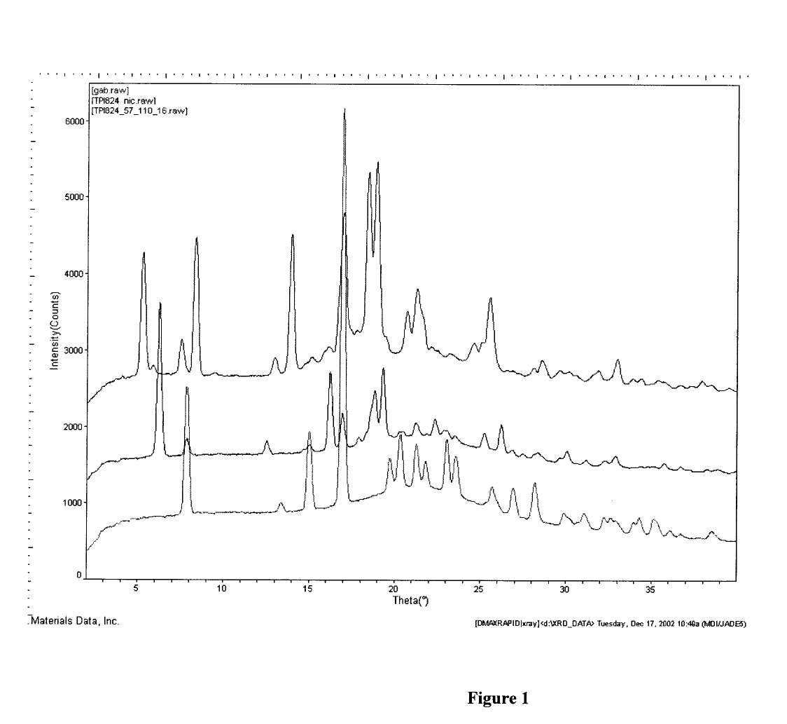

Solutions containing gabapentin (304.4 mg in 10 mL methanol) and DL-tartaric acid (1.1916 g in 5 mL methanol and 5 mL water) were prepared. The two solutions were mixed (100 microliters of the gabapentin solution and 2 microliters of the DL-tartaric acid solution) in a 2 mL vial and heated to 75 deg. C for 90 minutes. The sample was cooled to room temperature and then cooled to 5 deg. C and allowed to evaporate resulting in a solid material that was analyzed by PXRD and found to be different from forms 1 and 2 of gabapentin and gabapentin monohydrate. The results are presented in FIG. 1, which shows PXRD patterns for gabapentin form 1 (bottom), a mixture of gabapentin monohydrate (middle) and gabapentin form 1 and gabapentin DL-tartaric acid salt (top).

### Example 2

### Preparation of Gabapentin DL-Tartaric Acid Salt from Solid Mixture

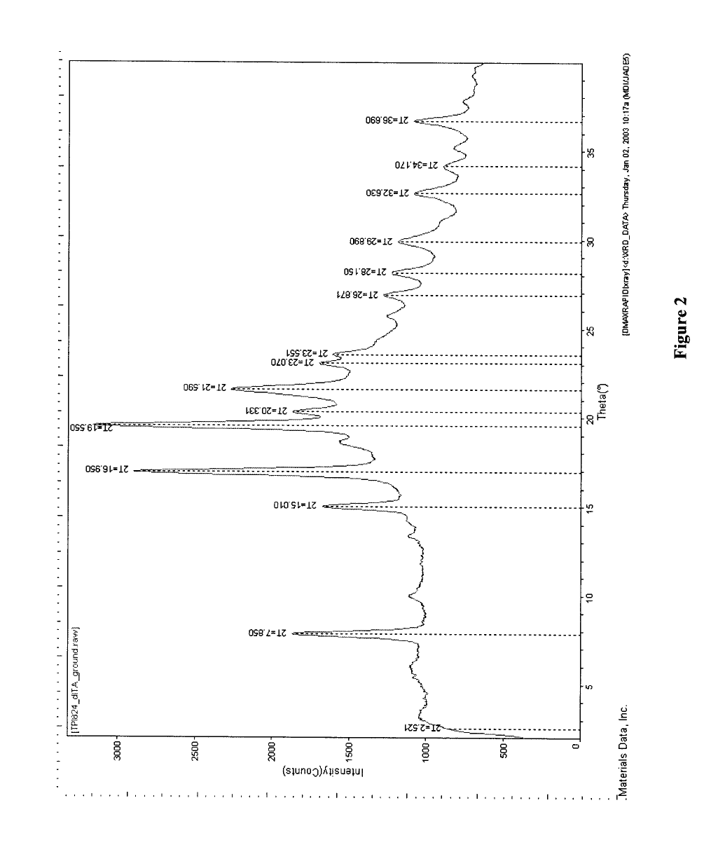

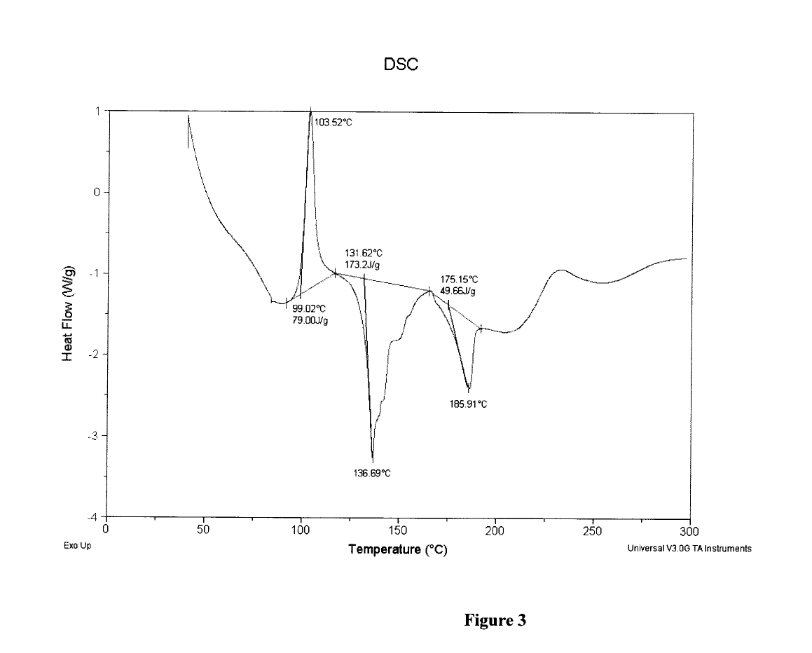

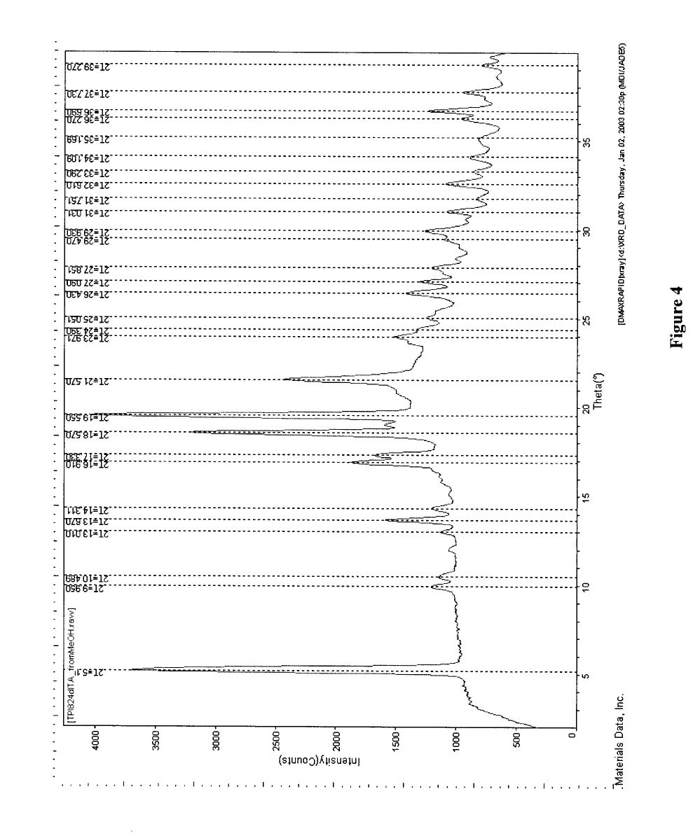

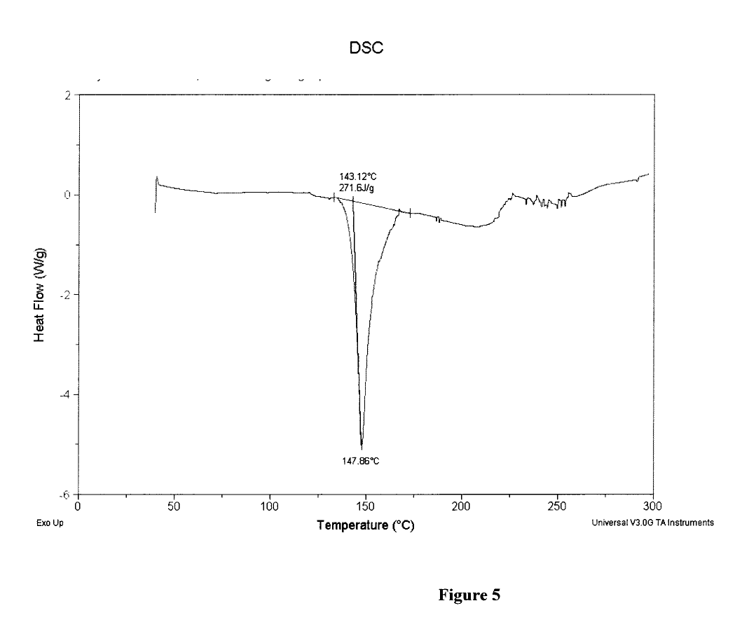

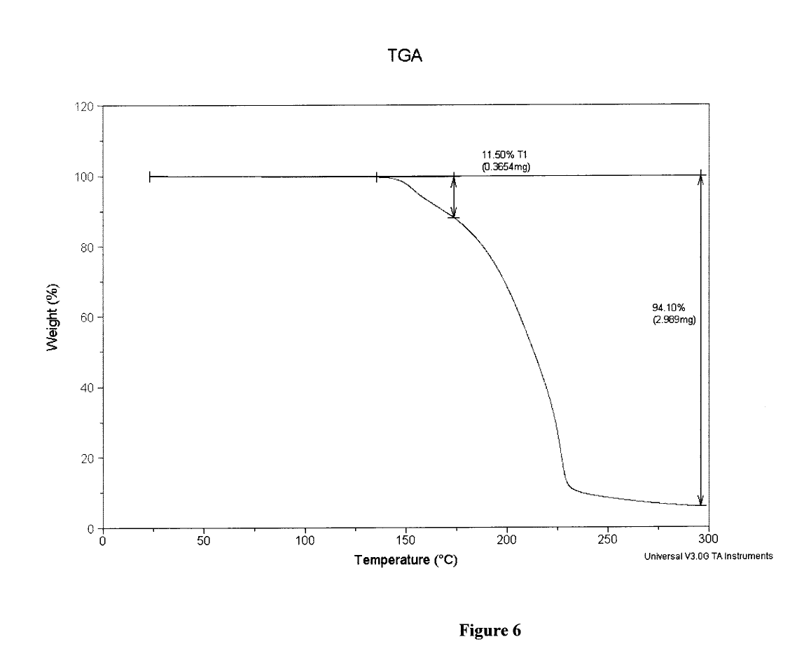

Gabapentin (98.07 mg) and DL-tartaric acid (90.2 mg) were deposited into a 20 mL scintillation vial. The solid mixture was transferred into a plastic capsule with a steel ball and ground for 3 minutes using a grinder. A portion of the resulting solid was analyzed by PXRD (FIG. 2) and DSC (FIG. 3). The resulting material was found to be a mixture of gabapentin and DL-tartaric acid. PXRD peaks include, but are not limited to, 7.85, 15.01, 16.95, 19.55, 20.33, 21.59, 23.07, 23.55, 26.87, 28.15, 29.89, and 32.63 degrees 2-theta. FIG. 3 shows a DSC trace of the solid resulting from the grinding of gabapentin and DL-tartaric acid. The exotherm at about 104 deg. C indicates that a salt is prepared from the two solids by heating them together. When heated, during the DSC experiment, the solid mixture underwent a crystallization event indicating that the salt between gabapentin and DL-tartaric acid was formed. The remaining unheated solid mixture was dissolved in methanol, filtered through a 2 micron filter into a 3 mL vial and the solvent was allowed to evaporate. Crystals grew, a small amount of which were ground and analyzed by PXRD (FIG. 4), DSC (FIG. 5) and TGA (FIG. 6). The form can be characterized by any one, any two, any three, any four, any five, or any six or more of the peaks in FIG. 4 including, but not limited to, 5.1, 9.95, 10.49, 13.01, 13.67, 14.31, 16.91, 17.33, 18.57, 19.55, 21.57, and 26.43 degrees 2-theta. The DSC thermogram (FIG. 5) shows an endothermic transition at about 148 deg. C. The TGA thermogram (FIG. 6) shows about a 11.5 weight loss between room temperature and about 175 deg. C. A single crystal structure was obtained using one of the unground crystals (Table 1) and showed that there was one gabapentin molecule for each DL-tartaric acid molecule.

TABLE 1
Unit Cell Parameters and Structural Information
for the Gabapentin DL-Tartaric Acid Salt

| Unit cell parameters | |
|---|---|
| a (Å) | 17.695(2) |
| b (Å) | 6.6547(8) |
| c (Å) | 13.3782(16) |
| α (°) | 90 |
| β (°) | 107.317(2) |
| γ (°) | 90 |
| V (Å³) | 1503.9(3) |
| Z | 4 |
| Crystal system | Monoclinic |
| Space group | P2(1)/c |
| Density (Mg/m³) | 1.419 |
| R1 | 0.0706 |
| wR2 | 0.1553 |

### Example 3

### Preparation of Gabapentin Ethanedisulfonic Acid Salt from Solution

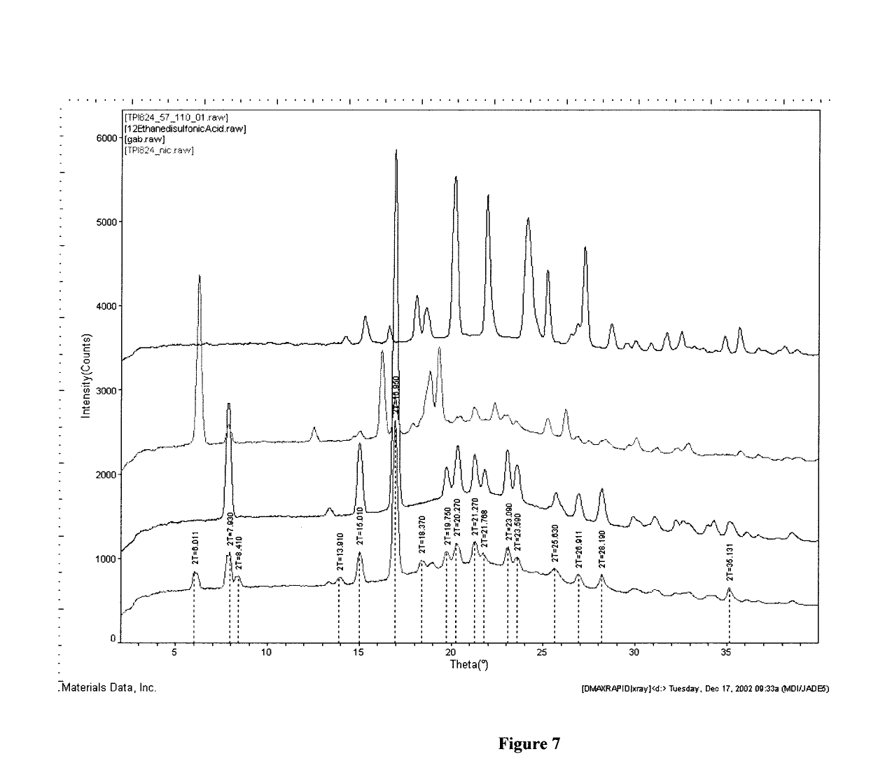

Solutions of gabapentin (304.44 mg in 10 mL of methanol) and ethanedisulfonic acid (0.92 g in 10 mL methanol) were prepared. To a vial was added 100 microliters of the gabapentin solution and 2 microliters of the ethanedisulfonic acid solution. The resulting solution was heated to 75 deg. C for 90 minutes in a capped vial, then cooled to room temperature and the vial cap was punctured. The solution was cooled to 5 deg. C and the solvent was allowed to evaporate. The resulting solid was analyzed by PXRD. FIG. 7 shows PXRD patterns for ethanedisulfonic acid (top), a mixture of gabapentin monohydrate and gabapentin form 1 (second from top), gabapentin form 1 (third from top) and the solid resulting from the evaporation of a methanolic solution containing ethanedisulfonic acid and gabapentin (bottom). The ethanedisulfonic acid gabapentin pattern (bottom) indicates a mixture of gabapentin form 1, gabapentin monohydrate and the ethanedisulfonic acid gabapentin salt is present. The bottom diffractogram from gabapentin and ethanedisulfonic acid has peaks including, but not limited to, 6.01, 7.93, 15.01, 16.95, 19.75, 20.27, 21.27, and 23.09 degrees 2-theta.

### Example 4

### Preparation of Gabapentin Ethanedisulfonic Acid Salt from Solid Mixture

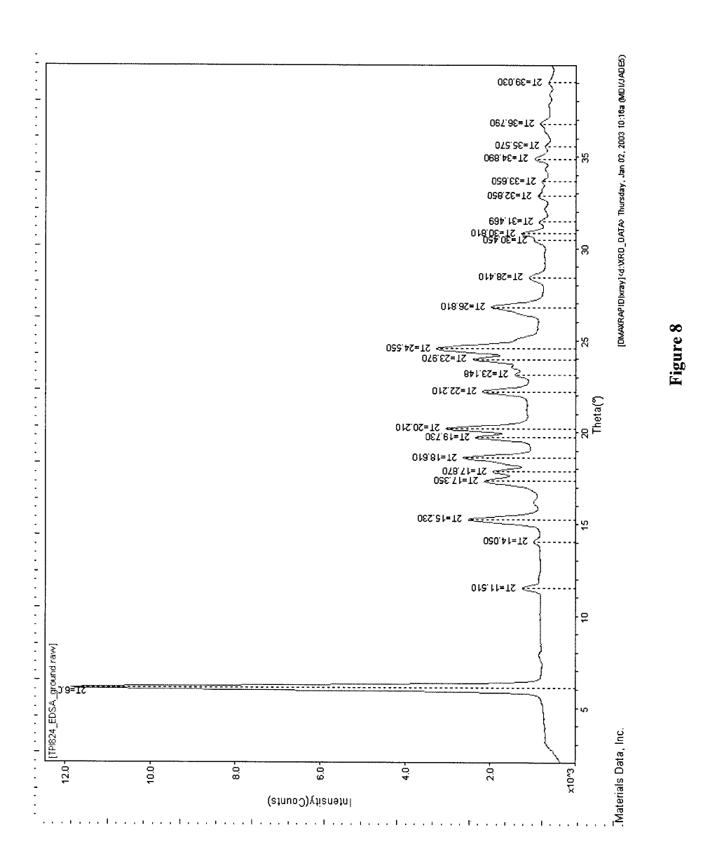

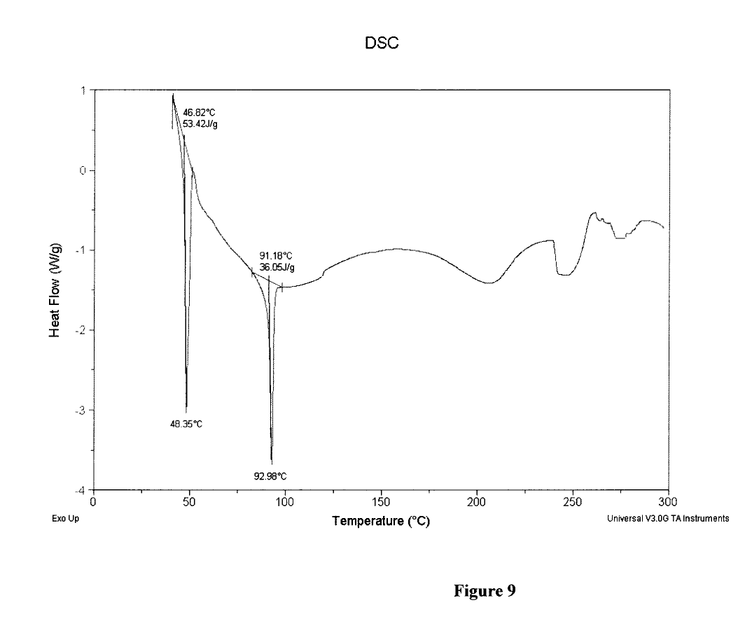

Gabapentin (103.0 mg) and ethanedisulfonic acid (1 17.9 mg) were deposited into a 20 mL scintillation vial. The solid mixture was transferred into a plastic capsule with a steel ball and ground for 3 minutes using a grinder. The resulting solid was analyzed by PXRD (FIG. 8) and DSC (FIG. 9). The solid was found to be a mixture of gabapentin and ethanedisulfonic acid. When heated, during the DSC experiment, the solid mixture underwent a crystallization event indicating that a gabapentin ethanedisulfonic acid salt was formed. FIG. 8 shows the PXRD pattern for the salt. The salt can be characterized by any one, any two, any three, any four, any five, or any six or more of the peaks in FIG. 8 including, but not limited to, 6.0, 11.51, 15.23, 17.35, 18.61, 19.73, 20.21, 22.21, 23.97, 24.55, and 26.81 degrees 2-theta. FIG. 9 shows a DSC thermogram for the salt showing two endotherms, one at about 48 deg. C and another at about 93 deg. C.

### Example 5

### Preparation of Gabapentin Ethanedisulfonic Acid Salt from Solution

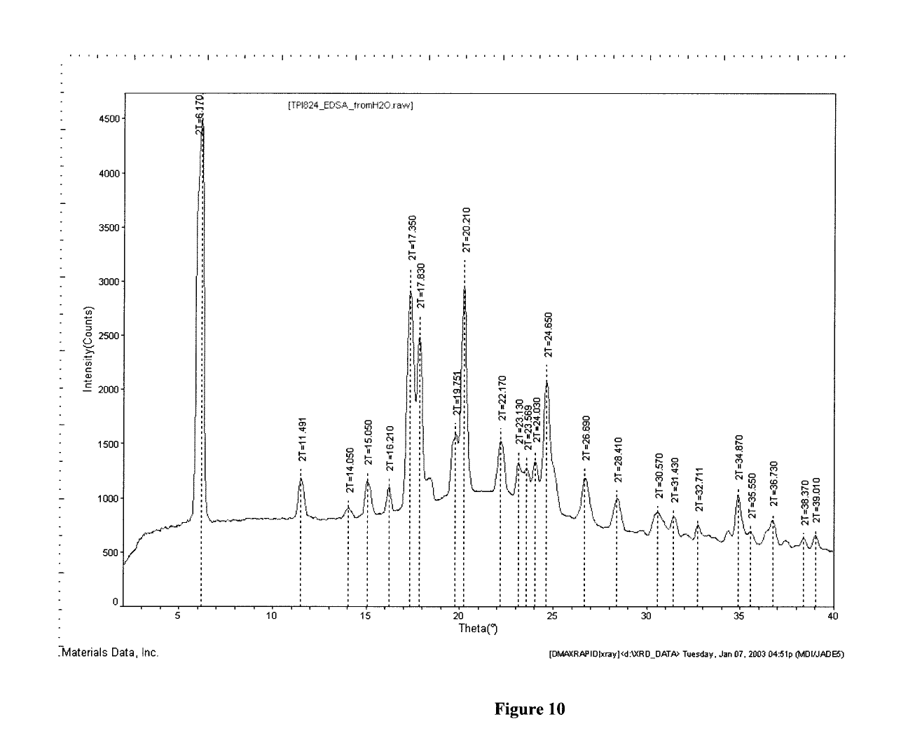

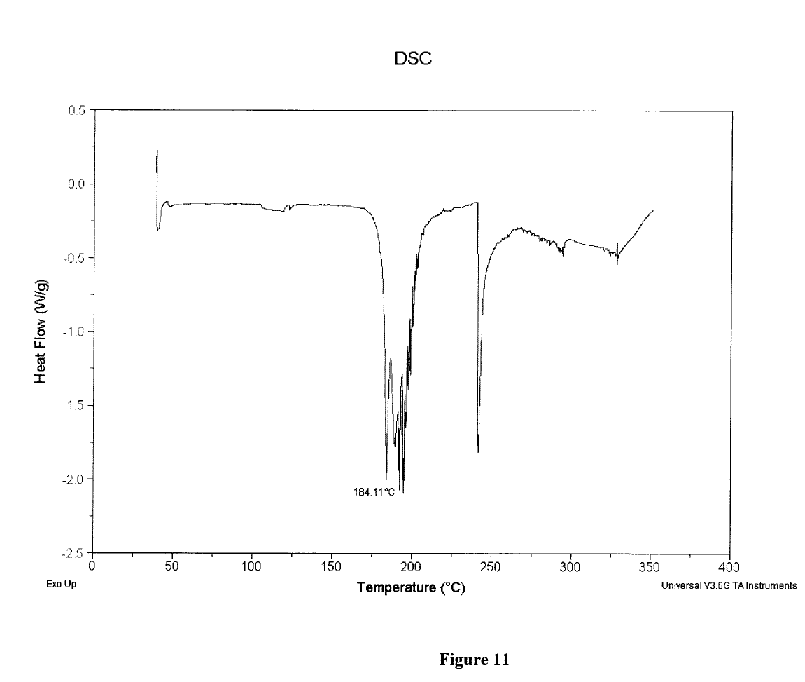

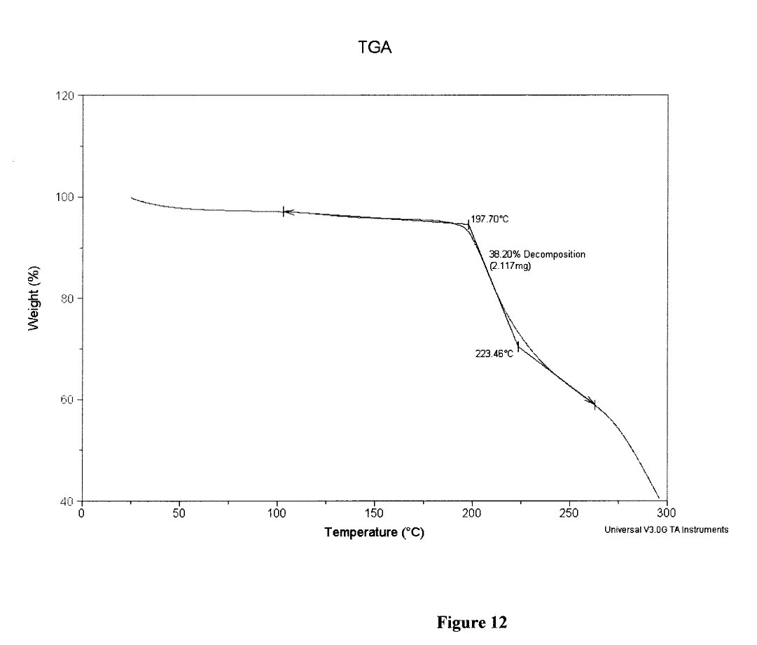

A solution of ethanedisulfonic acid was prepared from 0.92 g of ethanedisulfonic acid and 10 mL of methanol. Gabapentin (126.6 mg) and ethanedisulfonic acid (884 microliters, 0.837 M solution in methanol) were mixed in a 20 mL scintillation vial. 1 mL of water was added to the mixture and the solid gabapentin dissolved completely. The vial was covered with a piece of parafilm and the parafilm was punctured several times with a needle. The solvent was allowed to evaporate and the resulting crystals (needles) were analyzed by PXRD (FIG. 10), DSC (FIG. 11), TGA (FIG. 12) and single crystal X-ray diffraction (Table 2). The single crystal structure showed that there were two gabapentin molecules for each ethanedisulfonic acid molecule in the structure. FIG. 10 shows a PXRD pattern of the gabapentin ethanedisulfonic acid salt obtained by evaporation from a mixture of methanol and water. The salt can be characterized by any one, any two, any three, any four, any five, or any six or more of the peaks in FIG. 10 including, but not limited to, 6.17, 1 1.49, 15.05, 16.21, 17.35, 17.83, 20.21, 22.17, 24.65, 26.69, and 28.41 degrees 2-theta. FIG. 11 shows a DSC thermogram for gabapentin ethanedisulfonic acid salt showing an endothermic transition starting at about 184 deg. C. FIG. 12 shows a TGA thermogram for the gabapentin ethanedisulfonic acid salt where a weight loss of about 3 8 percent occured between about 100 deg. C and about 263 deg. C.

TABLE 2
Unit Cell Parameters and Structural Information
for the Gabapentin Ethanedisulfonic Acid Salt

| Unit cell parameters | |
|---|---|
| a (Å) | 5.5971 (7) |
| b (Å) | 8.0151 (10) |
| c (Å) | 14.6776 (18) |
| α (°) | 78.971 (2) |
| β (°) | 88.025 (2) |
| γ (°) | 75.867 (2) |
| V (Å³) | 626.68 (13) |
| Z | 2 |
| Crystal system | Triclinic |
| Space group | P(−1) |
| Density (Mg/m³) | 1.411 |
| R1 | 0.0632 |
| wR2 | 0.1446 |

### Example 6

### Preparation of Gabapentin Ethanedisulfonic Acid Salt from Aqueous Solution

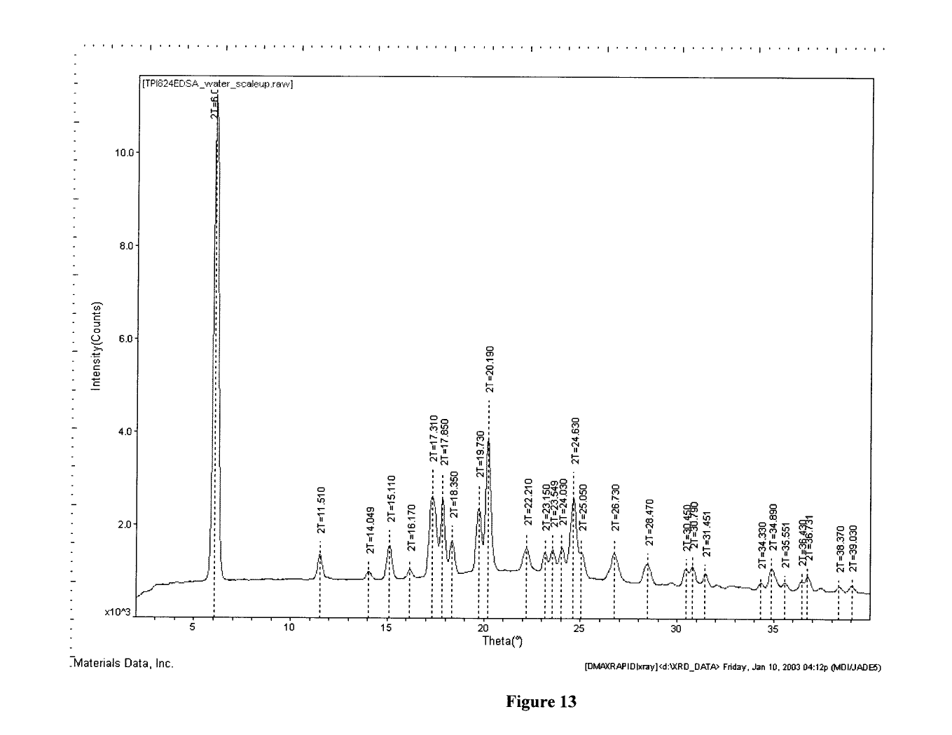

Gabapentin (0.55857 g) and ethanedisulfonic acid (0.62940 g) were transferred into a 20 mL scintillation vial. Water (1.5 mL) was added and the mixture was heated to 85 deg. C to dissolve all of the solids. The solution was filtered through a 0.2 micron filter into a new 20 mL scintillation vial. The vial was capped and placed in the refrigerator (2-4 deg. C) for 2 hours. The resulting crystals were collected by vacuum filtration (345.83 mg) and analyzed by PXRD. Upon analysis, the crystals were determined to be gabapentin ethanedisulfonic acid salt. A small amount of the crystalline salt (14.00 mg) was weighted into a 3 mL vial and 140 microliters of water was added. The pH of the resulting solution was determined to be 1.2. FIG. 13 shows the PXRD pattern for the gabapentin ethanedisulfonic acid salt. The salt can be characterized by any one, any two, any three, any four, any five, or any six or more of the peaks in FIG. 13 including, but not limited to, 6.0, 11.51, 15.11, 16.17, 17.31, 17.85, 19.73, 20.19, 22.21, 24.63, 26.73, and 28.47 degrees 2-theta.

### Example 7

### Preparation of Gabapentin Maleic Acid Salt from Solid Mixture

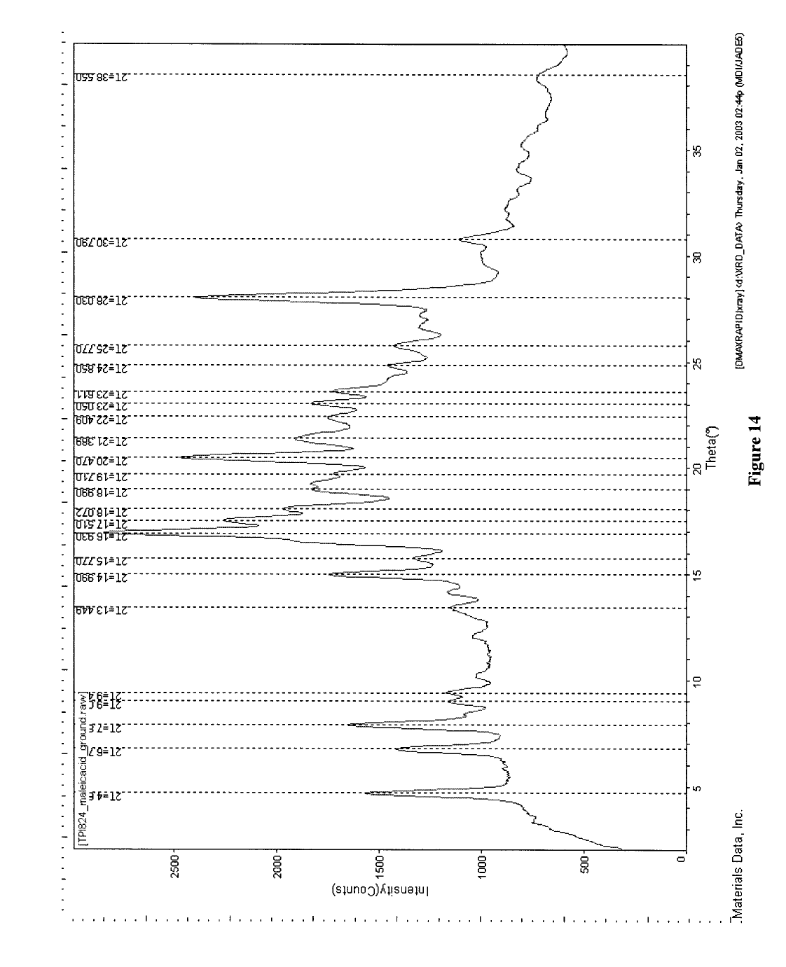

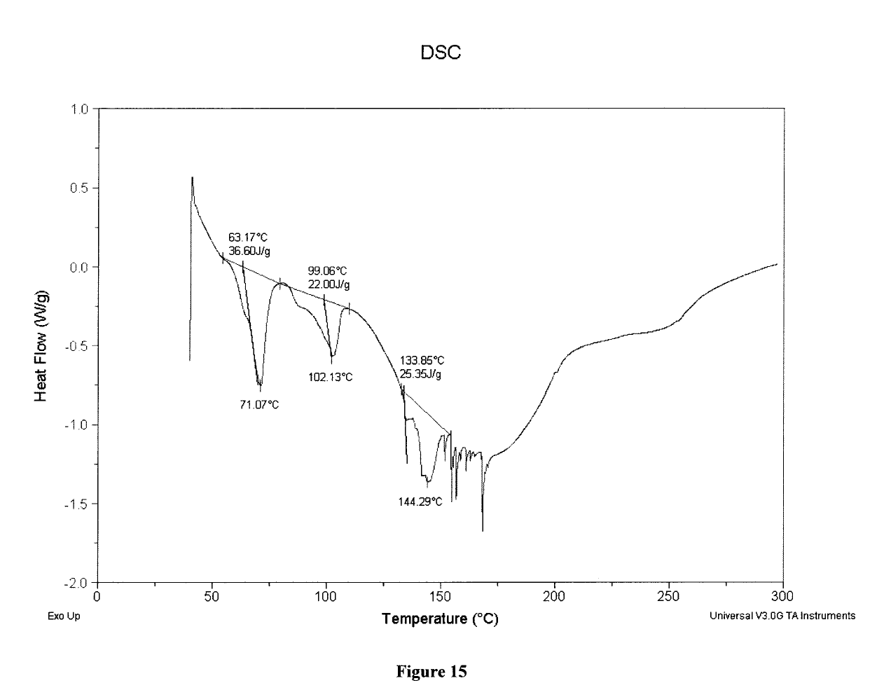

Gabapentin (103.0 mg) and maleic acid (70.8 mg) were deposited into a 20 mL scintillation vial. The solid mixture was transferred into a plastic capsule with a steel ball and ground for 3 minutes using a grinder. The resulting solid was analyzed by PXRD (FIG. 14) and DSC (FIG. 15). The solid was found to be a mixture of gabapentin and maleic acid. When heated, during the DSC experiment, the solid mixture underwent a crystallization event indicating that a gabapentin maleic acid salt was formed. FIG. 14 shows the PXRD diffractogram for the salt. The salt can be characterized by any one, any two, any three, any four, any five, or any six or more of the peaks in FIG. 14 including, but not limited to, 4.6, 6.7, 7.8, 9.0, 9.4, 13.45, 14.99, 16.93, 17.51, 18.07, 18.99, 20.47, 21.39, 23.05, 23.61, and 28.03 degrees 2-theta. FIG. 15 shows a DSC thermogram for the salt showing endotherms at about 71 deg. C and at about 102 deg. C.

### Example 8

### Preparation of Gabapentin:Urea Co-Crystal from Solution

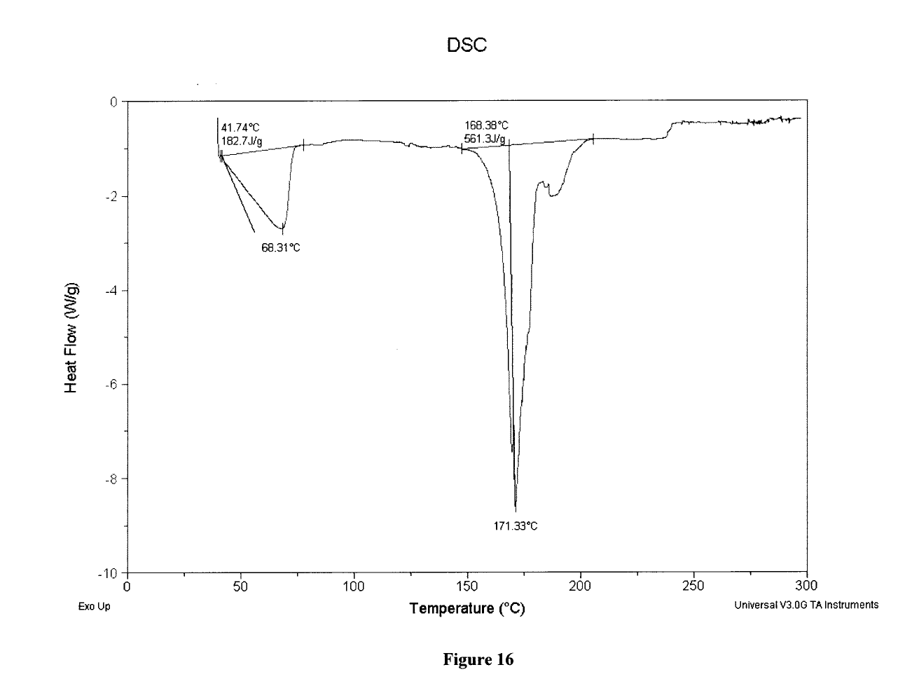

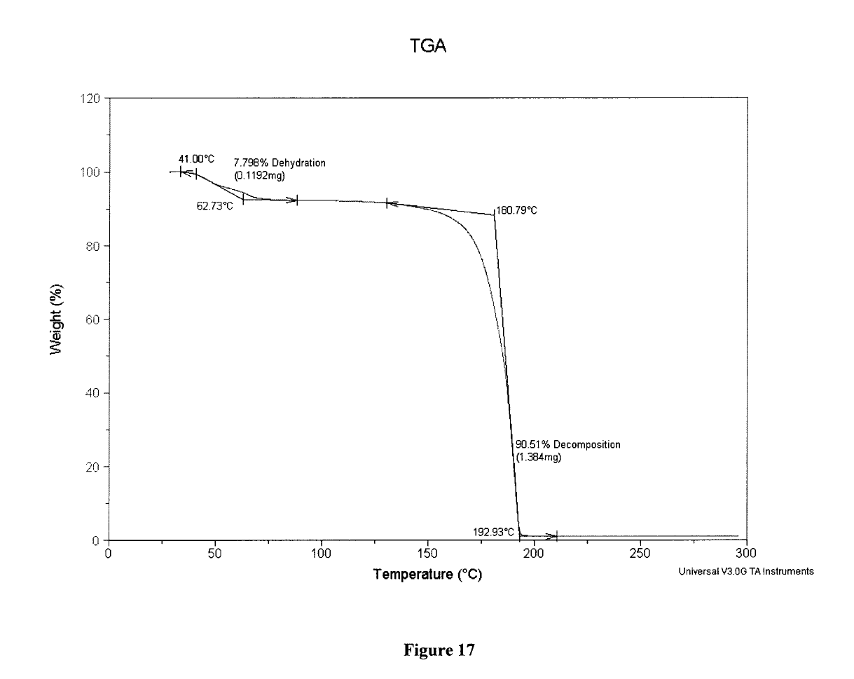

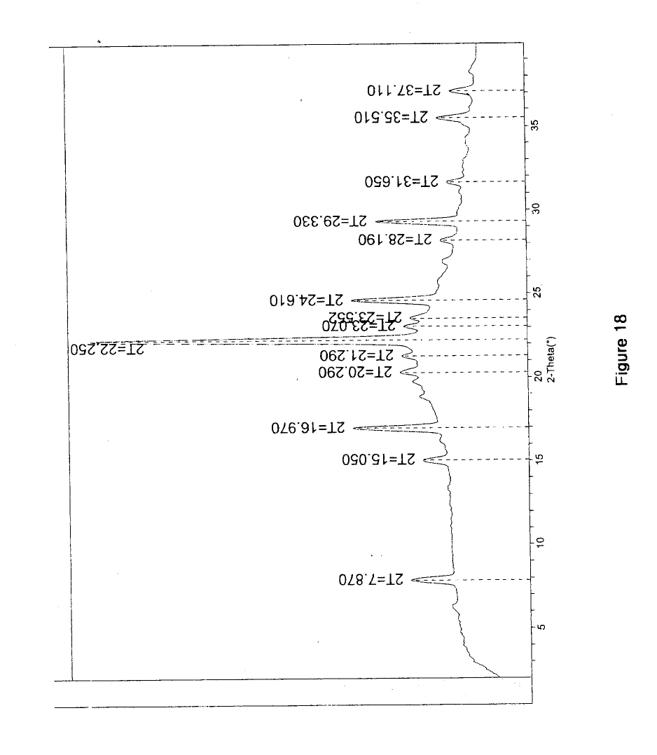

A solution of urea was prepared in methanol. Gabapentin (126.6 mg) and urea were mixed in a 20 mL scintillation vial. 1 mL of water was added to the mixture and the solid gabapentin dissolved completely. The vial was covered with a piece of parafilm and the parafilm was punctured several times with a needle. The solvent was allowed to evaporate and the resulting crystals were analyzed by DSC (FIG. 16), TGA (FIG. 17), and PXRD (FIG. 18). FIG. 16 shows a DSC thermogram for gabapentin urea co-crystal showing an endothermic transition at about 171 deg. C. FIG. 17 shows a TGA thermogram for the gabapentin urea co-crystal where a weight loss of about 7.8 percent occured between about room temperature and about 88 deg. C. The salt can be characterized by any one, any two, any three, any four, any five, or any six or more of the peaks in FIG. 18 including, but not limited to, 7.87, 15.05, 16.97, 22.25, 24.61, 29.33, 35.51, and 37.11 degrees 2-theta.

The term “co-crystal” as used herein means a crystalline material comprised of two or more unique solids at room temperature, each containing distinctive physical characteristics, such as structure, melting point, and heats of fusion, with the exception that, if specifically stated, the active pharmaceutical ingredient (API) may be a liquid at room temperature. A co-crystal is distinct from a salt due to the presence of neutral forms of the API and co-crystal former. Co-crystals are discussed further in U.S. application Ser. No. 10/660,202, which is incorporated by reference in its entirety.

While this invention has been particularly shown and described with references to preferred embodiments thereof, it will be understood by those skilled in the art that various changes in form and details may be made therein without departing from the scope of the invention encompassed by the appended claims.

1-21. (canceled)

22. A co-crystal comprising gabapentin and urea.

23. The co-crystal of claim 22, wherein said co-crystal is characterized by a powder X-ray diffraction pattern comprising peaks expressed in terms of 2-theta angles, and further wherein said X-ray diffraction pattern comprises peaks at 7.87, 16.97, and 22.25 degrees.

24. The co-crystal of claim 22, wherein said co-crystal is characterized by a powder X-ray diffraction pattern comprising peaks expressed in terms of 2-theta angles, and further wherein said X-ray diffraction pattern comprises peaks at 16.97, 24.61, and 29.33 degrees.

25. The co-crystal of claim 22, wherein said co-crystal is characterized by a powder X-ray diffraction pattern comprising peaks expressed in terms of 2-theta angles, and further wherein said X-ray diffraction pattern comprises peaks at 7.87, 24.61, and 29.33 degrees.

26. The co-crystal of claim 22, wherein said co-crystal is characterized by a powder X-ray diffraction pattern comprising peaks expressed in terms of 2-theta angles, and further wherein said X-ray diffraction pattern comprises peaks at 7.87 and 16.97 degrees.

27. The co-crystal of claim 22, wherein said co-crystal is characterized by a powder X-ray diffraction pattern comprising peaks expressed in terms of 2-theta angles, and further wherein said X-ray diffraction pattern comprises peaks at 16.97 and 22.25 degrees.

28. The co-crystal of claim 22, wherein said co-crystal is characterized by a powder X-ray diffraction pattern comprising peaks expressed in terms of 2-theta angles, and further wherein said X-ray diffraction pattern comprises peaks at 7.87 and 22.25 degrees.

29. The co-crystal of claim 22, wherein said co-crystal is characterized by a powder X-ray diffraction pattern comprising peaks expressed in terms of 2-theta angles, and further wherein said X-ray diffraction pattern comprises a peak at 7.87 degrees.

30. The co-crystal of claim 22, wherein said co-crystal is characterized by a powder X-ray diffraction pattern comprising peaks expressed in terms of 2-theta angles, and further wherein said X-ray diffraction pattern comprises a peak at 16.97 degrees.

31. The co-crystal of claim 22, wherein said co-crystal is characterized by a powder X-ray diffraction pattern comprising peaks expressed in terms of 2-theta angles, and further wherein said X-ray diffraction pattern comprises a peak at 22.25 degrees.

32. The co-crystal of claim 22, wherein said co-crystal is characterized by a DSC thermogram, and further wherein said DSC thermogram comprises an endothermic transition at about 171 degrees C.

33. The co-crystal of claim 22, wherein said co-crystal is characterized by a TGA thermogram, and further wherein said TGA thermogram comprises a weight loss of about 7.8 percent between about room temperature and about 88 degrees C.
